(19) United States
(12) Patent Application Publication
(10) Pub. No.: US 2019/0298661 A1
(43) Pub. Date: Oct. 3, 2019

(54) Transdermal Delivery of Phenethylamine Monoamine Releasers

(71) Applicant: The Corporation of Mercer University, Macon, GA (US)

(72) Inventors: Kevin S. Murnane, Norcross, GA (US); Ajay K. Banga, Duluth, GA (US); Sindhu S. Ganti, Atlanta, GA (US); Ying Jiang, Atlanta, GA (US); Ashana Puri, Atlanta, GA (US)

(21) Appl. No.: 16/346,180

(22) PCT Filed: Oct. 31, 2017
(86) PCT No.: PCT/US2017/059149

§ 371 Date: Apr. 30, 2019

Related U.S. Application Data
(60) Provisional application No. 62/415,108, filed on Oct. 31, 2016.
(60) Provisional application No. 62/519,592, filed on Jun. 14, 2017.

(51) Int. Cl. A61K 9/70; A61K 31/5375; A61K 31/4465; A61K 9/00

(52) U.S. Cl. CPC A61K 9/7038; A61K 9/0009; A61K 31/4465; A61K 31/5375

(57) ABSTRACT

Transdermal patches and transdermally applyable pharmaceutical compositions containing an effective amount of at least one drug are disclosed. The at least one drug may be: (a) phenmetrazine; (b) 4-benzylpiperidine; (c) 3-flouroamphetamine; (d) a DA/NE releaser compound having formula (I): wherein each of R₁, R₂, R₃, R₄, R₅, R₆, R₇, R₈, R₉ and R₁₀ is independently hydrogen, a halogen, a substituted or unsubstituted alkyl group, or a substituted or unsubstituted alkoxy group, and when one or more substituents is present on an alkyl group, an alkoxy group, or both, each substituent is independently a halogen; or (e) any combination thereof. Methods of making transdermal patches and transdermally applyable pharmaceutical compositions and methods of using transdermal patches and transdermally applyable pharmaceutical compositions are also disclosed.

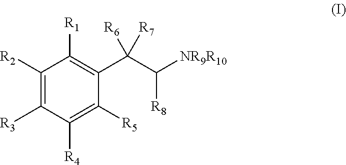

### CROSS REFERENCES TO RELATED APPLICATIONS

This application claims the priority benefit of U.S. Provisional Patent Application Nos. 62/415,108 and 62/519,592 filed Oct. 31, 2016 and Jun. 14, 2017, respectively. The entire contents of which are incorporated by reference.

### TECHNICAL FIELD

The present invention relates to therapies for treating disorders such as narcolepsy, Attention Deficit Hyperactivity Disorder (ADHD), lethargy, appetite suppression, and substance-use disorders (e.g., cocaine addiction).

### BACKGROUND

Efforts continue to further develop effective therapies for treating disorders such as narcolepsy, Attention Deficit Hyperactivity Disorder (ADHD), lethargy, appetite suppression, and substance-use disorders (e.g., cocaine addiction).

### SUMMARY

The present invention addresses some of the difficulties and problems discussed above by the discovery of new transdermal patches and pharmaceutical compositions containing one or more phenethylamine monoamine compounds.

Accordingly, in one exemplary embodiment, the present invention is directed to transdermal patches and pharmaceutical compositions containing one or more phenethylamine monoamine compounds. In one exemplary embodiment, the transdermal patches of the present invention comprise: a substrate; and an effective amount of at least one drug on and/or within said substrate, said at least one drug comprising: (a) phenmetrazine; (b) 4-benzylpiperidine; (c) 3-flouroamphetamine; (d) a dopamine/norepinephrine (DA/NE) releaser compound having formula (I):

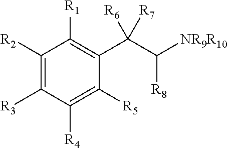

wherein each of R₁, R₂, R₃, R₄, R₅, R₆, R₇, R₈, R₉ and R₁₀ is independently hydrogen, a halogen, a substituted or unsubstituted alkyl group, or a substituted or unsubstituted alkoxy group, and when one or more substituents is present on an alkyl group, an alkoxy group, or both, each substituent is independently a halogen; or (e) any combination thereof.

The present invention is further directed to transdermally applyable pharmaceutical compositions comprising an effective amount of at least one drug, said at least one drug comprising: (a) phenmetrazine; (b) 4-benzylpiperidine; (c) 3-flouroamphetamine, (d) a DA/NE releaser DA/NE releaser compound having formula (I); or (e) any combination thereof. The pharmaceutical compositions may further comprise one or more additional components including, but not limited to, a solvent (e.g., water and/or one or more organic solvents).

The present invention is even further directed to methods of making transdermal patches and transdermally applyable pharmaceutical compositions. In one exemplary embodiment, the method of making a transdermal patch comprises forming a substrate with an effective amount of at least one drug on and/or within the substrate, the at least one drug comprising: (a) phenmetrazine, (b) 4-benzylpiperidine, (c) 3-flouroamphetamine, (d) a DA/NE releaser compound having formula (I), or (e) any combination thereof. In some embodiments, the substrate comprises an adhesive layer. The methods of making a transdermal patch may further comprise one or more steps including, but not limited to, laminating a backing layer onto the substrate (e.g., an adhesive layer), and applying a release liner onto at least one major surface of the substrate (e.g., an adhesive layer).

In a further exemplary embodiment, the method of making a transdermally applyable pharmaceutical composition comprises forming a composition comprising an effective amount of at least one drug, the at least one drug comprising: (a) phenmetrazine, (b) 4-benzylpiperidine, (c) 3-flouroamphetamine, (d) a DA/NE releaser compound having formula (I), or (e) any combination thereof. The methods of making a transdermally applyable pharmaceutical composition may further comprise one or more steps including, but not limited to, adding to the composition, one or more components selected from a solvent (e.g., water and/or one or more organic solvents).

In certain embodiments, the present invention provides a method of treating disorders such as narcolepsy, Attention Deficit Hyperactivity Disorder (ADHD), lethargy, appetite suppression, substance-use disorders (e.g., cocaine addiction), using any drugs, compounds, small molecules, proteins, antibodies, nucleotides, and pharmaceutical compositions thereof, that treat or alleviate the treated disorder, and are capable of transdermal delivery.

The invention contemplates any conventional methods for formulation of pharmaceutical compositions as described above. Various additives, known to those skilled in the art, may be included in the formulations. For example, solvents, including relatively small amounts of alcohol, may be used to solubilize certain drug substances. Other optional additives include opacifiers, antioxidants, fragrance, colorant, gelling agents, thickening agents, stabilizers, surfactants and the like. Other agents may also be added, such as antimicrobial agents, to prevent spoilage upon storage, i.e., to inhibit growth of microbes such as yeasts and molds. Suitable antimicrobial agents are typically selected from the group consisting of the methyl and propyl esters of p-hydroxybenzoic acid (i.e., methyl and propyl paraben), sodium benzoate, sorbic acid, imidurea, and combinations thereof.

Effective dosages and administration regimens can be readily determined by good medical practice and the clinical condition of the individual subject. The frequency of administration will depend on the pharmacokinetic parameters of the active ingredient(s), the specific method of transdermal administration, and desired therapeutic dosage. The optimal pharmaceutical formulation can be determined depending upon the specific method of transdermal administration and the desired dosage. Such formulations may influence the physical state, stability, rate of in vivo release, and rate of in vivo clearance of the administered compounds.

Depending on the specific method of transdermal administration, a suitable dose may be calculated according to body weight, body surface area, or organ size. Optimization of the appropriate dosage can readily be made by those skilled in the art in light of pharmacokinetic data observed in human clinical trials. The final dosage regimen will be determined by the attending physician, considering various factors which modify the action of drugs, e.g., the drug's specific activity, the severity of the damage and the responsiveness of the patient, the age, condition, body weight, sex and diet of the patient, the severity of the disorder(s) being treated, time of administration and other clinical factors.

The present invention is even further directed to a method of delivering one or more drugs to a patient, wherein the method comprises administering an effective amount of any one of the herein-described phenethylamine monoamine compounds via a transdermal patch or a transdermally applyable pharmaceutical composition to the patient. The methods of delivering one or more drugs to a patient may be used to treat a patient with one or more disorders, the one or more disorders comprising narcolepsy, Attention Deficit Hyperactivity Disorder (ADHD), lethargy, appetite suppression, substance-use disorders (e.g., cocaine addiction), or any combination thereof,

These and other features and advantages of the present invention will become apparent after a review of the following detailed description of the disclosed embodiments and the appended claims.

### BRIEF DESCRIPTION OF THE FIGURES

FIGS. 1A-1B depict the average cumulative amount of the base form of 4-benzylpiperidine per square centimeter that permeated across human dermatomed skin over 24 hours and show that the base form of 4-benzylpiperidine readily penetrates the skin and yields doses within the expected therapeutic range;

FIG. 2 depicts the average cumulative amount of the salt form of PAL-353 (3-flouroamphetamine) per square centimeter that permeated across human dermatomed skin over 24 hours using different enhancement techniques and show that iontophoresis enhancement allowed the salt form of PAL-353 (3-flouroamphetamine) to readily penetrate the skin and yield doses within the expected therapeutic range;

FIG. 3 depicts the average cumulative amount of the base form of PAL-353 (3-flouroamphetamine) per square centimeter that passively permeated across human dermatomed skin over 24 hours and shows that the base form of PAL-353 (3-flouroamphetamine) concentration-dependently and readily penetrates the skin and yields doses within the expected therapeutic range;

FIG. 4 depicts the average cumulative amount of the base form of phenmetrazine per square centimeter that passively permeated across human dermatomed skin over 24 hours and shows that the base form of phenmetrazine concentration-dependently and readily penetrates the skin; and

FIG. 5 depicts exemplary method steps for casting of an exemplary transdermal patch.

FIG. 6 depicts the permeation profiles of phenmetrazine base at 40 mg/mL in PG alone and PG with 5% w/w oleic acid, 10% w/w oleyl alcohol and 10% w/w lauric acid. (Student t-test: *, P<0.05; **, P<0.005; ***, P<0.001; ****, P<0.0001).

FIG. 7 depicts the permeation profiles of 3-FA base at 20 mg/mL in PG alone and PG with 5% w/w oleic acid, 10% w/w oleyl alcohol and 10% w/w lauric acid. (Student t-test: *, P<0.05; **, P<0.005; ***, P<0.001; ****, P<0.0001)

FIG. 8 depicts the fluxes over 24 hours of phenmetrazine at 40 mg/mL in PG alone and PG with 5% w/w oleic acid, 5% and 10% w/w oleyl alcohol and 10% w/w lauric acid.

FIG. 9 depicts the fluxes over 24 hours of PAL-353 (3-flouroamphetamine) at 20 mg/mL in PG alone and PG with 5% w/w oleic acid, 5% and 10% w/w oleyl alcohol and 10% w/w lauric acid.

FIG. 10 depicts the effect of chemical and physical enhancement techniques on the permeation of PAL-353 (3-flouroamphetamine) in PG across dermatomed human skin. (Student t-test: *, P<0.05, and ***, P<0.001)

FIG. 11 depicts the effect of iontophoresis on the permeation of PAL-353 (3-flouroamphetamine) in PBS across dermatomed human skin. (Student t-test: *, P<0.05)

### DETAILED DESCRIPTION

To promote an understanding of the principles of the present invention, descriptions of specific embodiments of the invention follow and specific language is used to describe the specific embodiments. It will nevertheless be understood that no limitation of the scope of the invention is intended by the use of specific language. Alterations, further modifications, and such further applications of the principles of the present invention discussed are contemplated as would normally occur to one ordinarily skilled in the art to which the invention pertains.

Additional embodiments are discussed below.

Transdermal Patches and Pharmaceutical Compositions Embodiments:

1. A transdermal patch comprising: a substrate; and an effective amount of at least one drug on and/or within said substrate, said at least one drug comprising: (a) phenmetrazine; (b) 4-benzylpiperidine; (c) 3-flouroamphetamine, (d) a DA/NE releaser compound having formula (I):

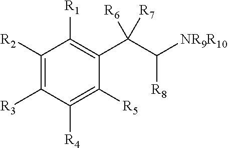

wherein each of R₁, R₂, R₃, R₄, R₅, R₆, R₇, R₈, R₉ and R₁₀ is independently hydrogen, a halogen, a substituted or unsubstituted alkyl group, or a substituted or unsubstituted alkoxy group, and when one or more substituents is present on an alkyl group, an alkoxy group, or both, each substituent is independently a halogen; or (e) any combination thereof.
2. The transdermal patch of embodiment 1, wherein said substrate comprises an adhesive layer.
3. The transdermal patch of embodiment 2, wherein said substrate further comprises a backing layer positioned on at least one major surface of said adhesive layer.
4. The transdermal patch of embodiment 3, wherein said backing layer comprises a film layer, a fibrous layer, or any combination thereof.
5. The transdermal patch of any one of embodiments 2 to 4, wherein said substrate further comprises a release liner positioned on at least one major surface of said adhesive layer.
6. The transdermal patch of any one of embodiments 2 to 5, wherein said adhesive layer comprises an acrylate (e.g., Duro-Tak 87-4098 and/or Duro-Tak 87-202A), a polyisobutylene (e.g., Duro-Tak 87-608A), a silicone, or any combination thereof.
7. The transdermal patch of any one of embodiments 1 to 6, wherein said at least one drug is within said substrate.
8. The transdermal patch of any one of embodiments 2 to 7, wherein said at least one drug is within said adhesive layer.
9. The transdermal patch of any one of embodiments 1 to 8, wherein said at least one drug is phenmetrazine.
10. The transdermal patch of any one of embodiments 1 to 9, wherein said at least one drug is 4-benzylpiperidine.
11. The transdermal patch of any one of embodiments 1 to 10, wherein said at least one drug is a DA/NE releaser compound having formula (I).
12. The transdermal patch of any one of embodiments 1 to 11, wherein said at least one drug is a DA/NE releaser compound having formula (I) and R₅ is hydrogen.
13. The transdermal patch of any one of embodiments 1 to 12, wherein said at least one drug is a DA/NE releaser compound having formula (I) and R₄ is hydrogen.
14. The transdermal patch of any one of embodiments 1 to 13, wherein said at least one drug is a DA/NE releaser compound having formula (I) and at least one of R₁, R₂ and R₃ is not hydrogen.
15. The transdermal patch of any one of embodiments 1 to 14, wherein said at least one drug is a DA/NE releaser compound having formula (I) and at least one of R₁, R₂ and R₃ is a halogen.
16. The transdermal patch of any one of embodiments 1 to 15, wherein said at least one drug is a DA/NE releaser compound having formula (I) and at least two of R₁, R₂ and R₃ is a halogen.
17. The transdermal patch of any one of embodiments 1 to 16, wherein said at least one drug is a DA/NE releaser compound having formula (I) and at least one of R₁, R₂ and R₃ is a halogen, and at least one of R₁, R₂ and R₃ is a substituted or unsubstituted alkyl group or a substituted or unsubstituted alkoxy group.
18. The transdermal patch of any one of embodiments 1 to 17, wherein said at least one drug is a DA/NE releaser compound having formula (I) and at least one of R₁, R₂ and R₃ is a halogen, and at least one of R₁, R₂ and R₃ is a substituted or unsubstituted alkyl group.
19. The transdermal patch of any one of embodiments 1 to 18, wherein said at least one drug is a DA/NE releaser compound having formula (I) and R₃ is a halogen.
20. The transdermal patch of any one of embodiments 1 to 19, wherein said at least one drug is a DA/NE releaser compound having formula (I) and R₂ is a halogen.
21. The transdermal patch of any one of embodiments 1 to 19, wherein said at least one drug is a DA/NE releaser compound having formula (I) and R₂ is a substituted or unsubstituted alkyl group.
22. The transdermal patch of any one of embodiments 1 to 19, wherein said at least one drug is a DA/NE releaser compound having formula (I) and R₂ is a substituted or unsubstituted alkoxy group.
23. The transdermal patch of any one of embodiments 1 to 18 and 20 to 22, wherein said at least one drug is a DA/NE releaser compound having formula (I) and R₃ is a substituted or unsubstituted alkyl group, or a substituted or unsubstituted alkoxy group.
24. The transdermal patch of any one of embodiments 1 to 18 and 20 to 23, wherein said at least one drug is a DA/NE releaser compound having formula (I) and R₃ is a substituted or unsubstituted alkyl group.
25. The transdermal patch of any one of embodiments 1 to 18 and 20 to 23, wherein said at least one drug is a DA/NE releaser compound having formula (I) and R₃ is a substituted or unsubstituted alkoxy group.
26. The transdermal patch of any one of embodiments 1 to 25, wherein said at least one drug is a DA/NE releaser compound having formula (I) and R₁ is a halogen.
27. The transdermal patch of any one of embodiments 1 to 25, wherein said at least one drug is a DA/NE releaser compound having formula (I) and R₁ is a substituted or unsubstituted alkyl group.
28. The transdermal patch of any one of embodiments 1 to 25, wherein said at least one drug is a DA/NE releaser compound having formula (I) and R₁ is a substituted or unsubstituted alkoxyl group.
29. The transdermal patch of any one of embodiments 1 to 28, wherein each of said halogen independently comprises chlorine or fluorine.
30. The transdermal patch of any one of embodiments 1 to 29, wherein each of said alkyl group or said alkoxyl group independently comprises from one to eight carbon atoms.
31. The transdermal patch of any one of embodiments 1 to 30, wherein each of said alkyl group or said alkoxyl group independently comprises from one to three carbon atoms.
32. The transdermal patch of any one of embodiments 1 to 31, wherein each of said alkyl group or said alkoxyl group independently comprises one carbon atom.
33. A transdermally applyable pharmaceutical composition, said pharmaceutical composition comprising an effective amount of at least one drug, said at least one drug comprising: (a) phenmetrazine; (b) 4-benzylpiperidine; (c) 3-flouroamphetamine; (d) a DA/NE releaser compound having formula (I) as recited in any one of embodiments 1 and 9 to 32, or (e) any combination thereof.
34. The transdermal patch or transdermally applyable pharmaceutical composition of any one of embodiments 1 to 33, wherein said effective amount of at least one drug comprises from greater than 0 to about 10 milligrams/kg/24 hour.
35. The transdermal patch or transdermally applyable pharmaceutical composition of any one of embodiments 1 to 34, wherein said effective amount of at least one drug comprises from greater than 0 to about 3 milligrams/kg/24 hour.

Methods of Making Transdermal Patches Embodiments:

36. A method of making the transdermal patch of any one of embodiments 1 to 32 and 34 to 35, said method comprising: forming the substrate with the effective amount of (a) phenmetrazine, (b) 4-benzylpiperidine, (c) 3-flouroamphetamine, (d) a DA/NE releaser compound having formula (I), or (e) any combination thereof on and/or in the substrate.
37. The method of embodiment 36, wherein said forming step comprises forming an adhesive layer with the effective amount of (a) phenmetrazine, (b) 4-benzylpiperidine, (c) 3-flouroamphetamine, (d) a DA/NE releaser compound having formula (I), or (e) any combination thereof on and/or in the adhesive layer.
38. The method of embodiment 37, wherein said forming step further comprises laminating a backing layer onto the adhesive layer.
39. The method of embodiment 37 or 38, wherein said forming step further comprises applying a release liner onto at least one major surface of the adhesive layer.

It should be noted that any of the above methods of making the herein-described transdermal patch may further comprise one or more method steps including, but not limited to, forming an emulsion, forming multiple adhesive layers, combining multiple layers with one another, or any combination thereof.

Methods of Making Transdermally Applyable Pharmaceutical Compositions Embodiments:

40. A method of making the transdermally applyable pharmaceutical composition of any one of embodiments 33 to 35, said method comprising: forming a composition with the effective amount of (a) phenmetrazine, (b) 4-benzylpiperidine, (c) 3-flouroamphetamine, (d) a DA/NE releaser compound having formula (I), or (e) any combination thereof.

Methods of Using Transdermal Patches and Transdermally Applyable Pharmaceutical Compositions to Deliver One or More Drugs to a Patient Embodiments:

41. A method of delivering one or more drugs to a patient, said method comprising: transdermally administering an effective amount of at least one drug comprising: (a) phenmetrazine, (b) 4-benzylpiperidine, (c) 3-flouroamphetamine, (d) a DA/NE releaser compound having formula (I), or (e) any combination thereof, as recited in any one of embodiments 1 and 9 to 32, to the patient.
42. The method of embodiment 41, wherein said transdermally administering step comprises applying the transdermal patch of any one of embodiments 1 to 32 and 33 to 35 onto the patient.
43. The method of embodiment 41, wherein said transdermally administering step comprises applying the transdermally applyable pharmaceutical composition of any one of embodiments 33 to 35 onto the patient.
44. The method of any one of embodiments 41 to 43, wherein said transdermally administering step further comprises utilizing one or more enhancement techniques, the one or more enhancement techniques comprising use of microneedles, chemical enhancement, laser ablation, iontophoresis, or any combination thereof.
45. The method of any one of embodiments 41 to 44, wherein said transdermally administering step further comprises utilizing one or more enhancement techniques, the one or more enhancement techniques comprising use of microneedles.
46. The method of any one of embodiments 41 to 45, wherein said transdermally administering step further comprises utilizing one or more enhancement techniques, the one or more enhancement techniques comprising use of chemical enhancement. The chemical enhancement may comprise oleic acid, oleyl alcohol, isopropyl myristate, lauric acid or any combination thereof.
47. The method of any one of embodiments 41 to 46, wherein said transdermally administering step further comprises utilizing one or more enhancement techniques, the one or more enhancement techniques comprising use of laser ablation.
48. The method of any one of embodiments 41 to 47, wherein said transdermally administering step further comprises utilizing one or more enhancement techniques, the one or more enhancement techniques comprising use of iontophoresis.
49. The method of any one of embodiments 41 to 48, wherein said method is used to treat a patient with one or more disorders, the one or more disorders comprising narcolepsy, Attention Deficit Hyperactivity Disorder (ADHD), lethargy, appetite suppression, substance-use disorders (e.g., cocaine addiction), or any combination thereof.
50. The method of any one of embodiments 41 to 49, wherein said method is used to treat a patient with one or more disorders, the one or more disorders comprising narcolepsy.
51. The method of any one of embodiments 41 to 50, wherein said method is used to treat a patient with one or more disorders, the one or more disorders comprising Attention Deficit Hyperactivity Disorder (ADHD).
52. The method of any one of embodiments 41 to 51, wherein said method is used to treat a patient with one or more disorders, the one or more disorders comprising lethargy.
53. The method of any one of embodiments 41 to 52, wherein said method is used to treat a patient with one or more disorders, the one or more disorders comprising appetite suppression.
54. The method of any one of embodiments 41 to 53, wherein said method is used to treat a patient with one or more disorders, the one or more disorders comprising substance-use disorders (e.g., cocaine addiction).
55. The method of any one of embodiments 41 to 54, wherein said method is used to treat a patient with one or more disorders, the one or more disorders comprising cocaine addiction.
56. The method of any one of embodiments 36 to 55, wherein the effective amount of at least one drug comprises from greater than 0 to about 10 milligrams/kg/24 hour.
57. The method of any one of embodiments 36 to 56, wherein the effective amount of at least one drug comprises from greater than 0 to about 3 milligrams/kg/24 hour.

The present invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLES

### Example 1—Preparation and Use of Transdermal Patches and Pharmaceutical Compositions

In this example of the present invention, transdermal patches and pharmaceutical compositions comprising phenethylamine analogs shown in Table 1 below were prepared.

TABLE 1
Release activity of phenethylamine analogs

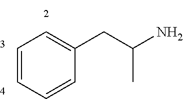

| | | EC₅₀, (nM)^{a} | | | SERT/ | SERT/ |
|---|---|---|---|---|---|---|
| PAL# | Substituent | DAT | SERT | NET | DA | NET |
| 10 | Piperidine | | | | | |
| (4-Benzylpiperidine) | | | | | | |
| 56 | Morpholine | | | | | |
| (Phenmetrazine) | | | | | | |
| 353 (3- | 3-F | 24.2 | 1937 | 16.1 | 80 | 120 |
| Flouroamphetamine) | | | | | | |
| 313 | 4-CH₃ | 44.1 | 53.4 | 22.2 | 1.2 | 2.4 |
| 24 | 4-Cl | 68.5 | 21.5 | 23.5 | 0.3 | 0.9 |
| 303 | 4-F | 51.5 | 939 | 28.0 | 18.2 | 33.5 |
| 162 | 4-OCH₃ | 867 | 63.3 | 166 | 0.1 | 0.4 |
| 309 | 4-OCF₃ | 2190 | 82.2 | 454 | 0.0 | 0.2 |
| 335 | 4-Et | 400 | 36.1 | 127 | 0.1 | 0.3 |
| 334 | 4-iPr | >10 k | 79.2 | 3364 | | — |
| 314 | 3-CH₃ | 33.3 | 218 | 18.3 | 6.5 | 11.9 |
| 677 | 3-CF₃ | 1900^{a} | 104^{a} | 170^{a} | 0.1 | 0.6 |
| 304 | 3-Cl | 11.8 | 120 | 9.4 | 10.2 | 12.8 |
| 161 | 3-OCH₃ | 103 | 328 | 58.0 | 3.2 | 5.7 |
| 1386 | 3-Et | 127 | 160 | 111 | 1.3 | 1.4 |
| 315 | 2-CH₃ | 127 | 973 | 37.0 | 7.7 | 26.3 |
| 307 | 2-CF₃ | 2743 | >10 k | 640 | | — |

The physiochemical properties of PAL-353 (MW 190 (Hydrochloride salt) or MW 153 (Base form); log P 1.95—www.Chemicalize.com), phenmetrazine (MW 293 (Fumerate salt) or MW 177 (Base form); log P 1.79—www.Chemicalize.com), phendimetrazine (MW 213; log P 2.17—www.chemspider.com), and 4-benzylpiperidine (MW 175; log P 2.52—www.Chemicalize.com) suggested that these small and moderately lipophilic molecules should partition well into the skin. To test this prediction, skin permeation studies were conducted with 4-benzylpiperidine, phenmetrazine, and PAL-353 (3-flouroamphetamine) Based on the required dosing and technical capacity of patch technology, one objective was to develop a 24-hour patch. As such, the skin permeation studies were performed for 24 hours using vertical static Franz-type diffusion cells.

The recirculating water bath system was maintained at 37° C. to bring the skin surface temperature to 32° C. Each Franz cell comprised of a donor compartment containing the drug and a receiver compartment containing 1×PBS at pH 7.4 to ensure a diffusion gradient, with human dermatomed skin (described below) clamped between the two compartments. Permeation was followed by detection of drug in the receiver compartment using an alliance HPLC Waters 2695 Separations Module attached to a Waters UV detector system. A prodigy 5 u ODS (2) 150×4.60 mm 5 micron column with mobile phase of acetonitrile and water (0.05% TFA) at a flow rate of 1 mL/min and an injection volume 20 μL, with a retention time of 5.5 min, was detected at 259 nm wavelength. The same permeation and HPLC protocol was followed with 6 Franz cells (n=6) for each condition.

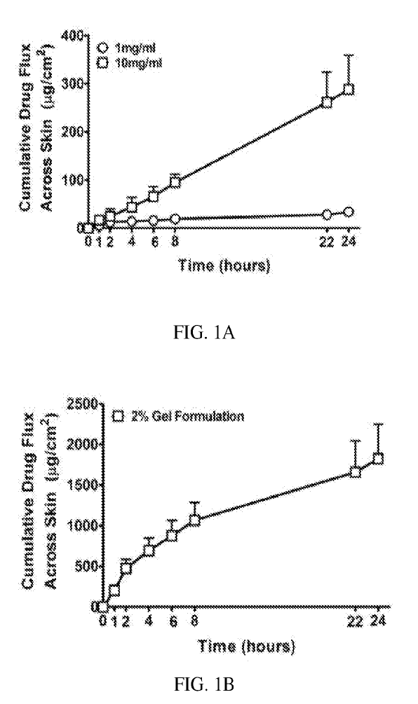

In the first permeation study, 100 μL of 10 mg/ml and 1 mg/ml of 4-benzylpiperidine liquid base form in PG (propylene glycol) was added to the donor and an average of 288±71 μg/cm² and 33±8 μg/cm² respectively permeated across the dermatomed human skin (as seen in FIG. 1A). 4-benzylpiperidine was then made into a gel formulation with hydroxyl propyl cellulose (HPC) at varying concentrations (1.5%, 2% and 4% HPC), PG, and water. As it exhibited the highest stability, the permeation study was performed using 100 mg of 2% HPC gel that contained 10 mg of drug out of which, about 18% (average of 1824±425 μg/cm²) of drug permeated across the human dermatomed skin (FIG. 1B). These data demonstrate that 4-benzylpiperidine exhibited excellent passive skin permeation without the need for enhancement and yields doses within the expected therapeutic range.

To demonstrate skin permeation of another dopamine/norepinephrine (DA/NE) releaser, the study shifted from 4-benzylpiperidine to the PAL-353 (3-flouroamphetamine) PAL-353 (3-flouroamphetamine) skin permeation study conditions were similar to those outlined for 4-benzylpiperidine above. PAL-353 (3-flouroamphetamine) (10 mg/mL in a vehicle suitable for the enhancement technique being used) was placed in the donor compartment of the Franz cell.

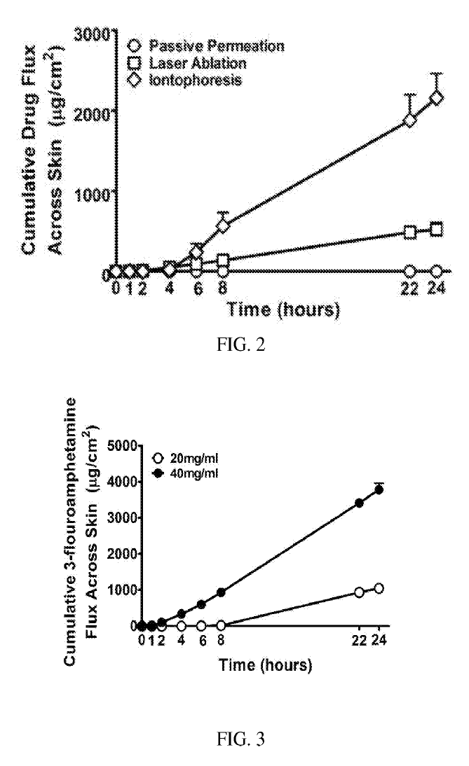

The salt form of PAL-353 (3-flouroamphetamine) was initially studied. In contrast to 4-benzylpiperidine, PAL-353 (3-flouroamphetamine) exhibited relatively low levels of skin permeation using passive delivery. A series of studies were conducted using state-of-the art permeation enhancement techniques available in the Banga laboratory (Mercer University, Atlanta Ga.), including microneedles, oleic acid chemical enhancement, laser ablation, and iontophoresis. See, FIG. 2. Each of these techniques produced measurable increases in skin flux of the salt form of PAL-353 (3-flouroamphetamine); however, iontophoresis enhancement engendered significantly higher flux than any of the other techniques. Importantly, the calculated flux using iontophoresis resulted in the delivery of 2159.43±301.14 μg of PAL-353/cm² over 24 hours.

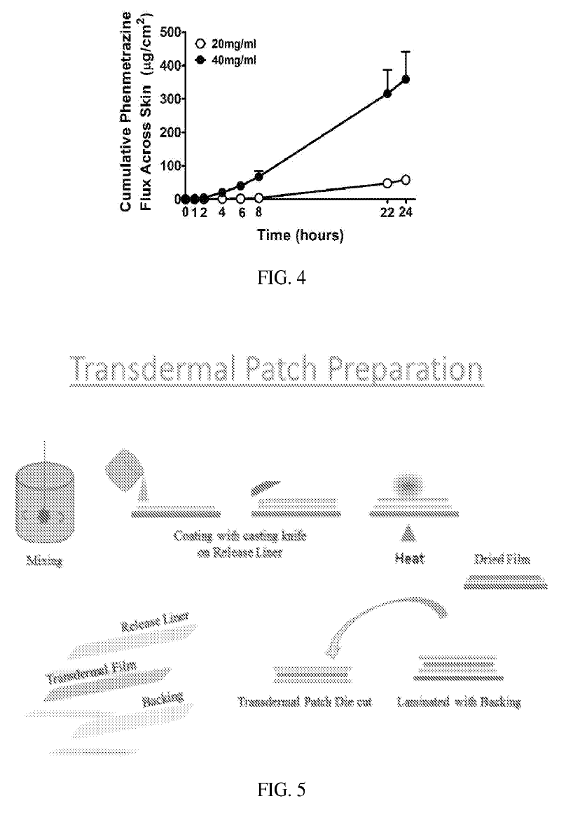

The skin permeation of the base forms of PAL-353 and phenmetrazine were then tested to determine whether the flux of each compound depended on the concentration applied transdermally. The results of these experiments showed that the base form of PAL-353 (FIG. 3) and phenmetrazine (FIG. 4) passively penetrate the skin in a concentration dependent manner. The calculated passive flux of the base form of PAL-353 at a concentration of 40 mg/ml was 3784.62±173.52 μg/cm² over 24 hours. The calculated passive flux of phenmetrazine at a concentration of 40 mg/ml was 359.05±82.36 μg/cm² over 24 hours. It is likely that this level of permeation of phenmetrazine can be increase further by increasing the available concentration or through chemical or active enhancement.

### Example 2—Transdermal Permeation

The permeation of phenmetrazine and PAL-353 (3-flouroamphetamine) (3-FA) through dermatomed human cadaver skin was further studied using static Franz diffusion cells. Permeated drugs were quantified using a validated HPLC-UV method. The passive permeation of phenmetrazine and 3-FA was evaluated at the concentrations of 10 (phenmetrazine fumarate), 20 (phenmetrazine and 3-FA bases) and 40 (phenmetrazine and 3-FA bases) mg/mL in propylene glycol (PG). Further, the enhancement effect of oleic acid (5% w/w), oleyl alcohol (5% and 10% w/w) and lauric acid (10% w/w) on the permeation of phenmetrazine and 3-FA bases at 20 and 40 mg/mL was investigated.

Phenmetrazine fumarate showed no permeation over 24 hours. Within 24 hours, 1.86±0.41% and 5.74±1.32% of phenmetrazine base permeated through the skin from 20 and 40 mg/mL groups, respectively. 3-FA base showed high passive permeation with 41.4±1.18% and 74.45±3.43% permeated from 20 and 40 mg/mL groups over 24 hours. Compared to the propylene glycol only group, 10% oleyl alcohol, 5% oleic acid, and 10% lauric acid enhanced the flux of phenmetrazine base by 11.4, 7.6 and 3.4 fold, respectively, with 10% oleyl alcohol and 5% oleic acid groups achieving the required flux for therapeutically relevant delivery. Oleyl alcohol (10%) and oleic acid (5%) increased the flux of 3-FA base to 292.0±31.70 and 109.8±17.94 μg/cm²/h, while 10% lauric acid group did not show significant difference with the propylene glycol alone group. Furthermore, at 5%, oleyl alcohol showed significantly (P<0.001) higher cumulative amount and flux than the oleic acid for both bases. However, 5% and 10% oleyl alcohol showed no significant difference in the flux of both bases. Two formulations reaching therapeutic flux were predicted to be irritant for phenmetrazine and irritant for 3-FA.

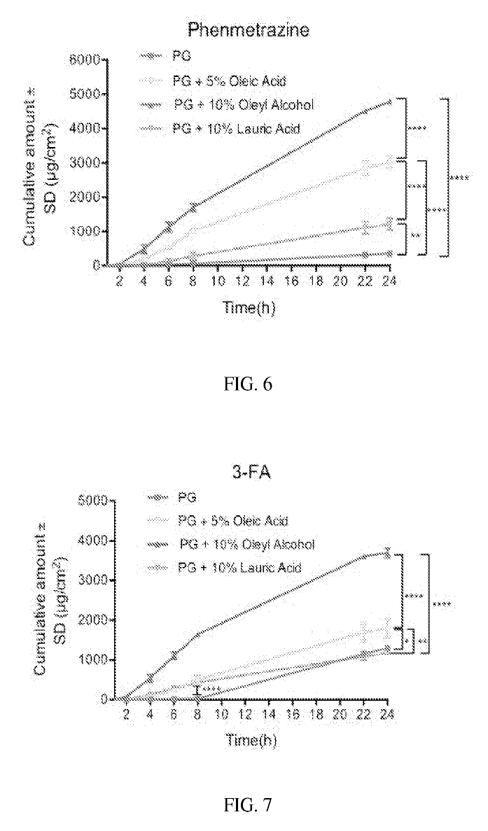

The permeation profiles of phenmetrazine base at 40 mg/mL (FIG. 6) and 3-FA base at 20 mg/mL (FIG. 7) in PG alone and PG with 5% w/w oleic acid, 10% w/w oleyl alcohol and 10% w/w lauric acid. (Student t-test: *, P<0.05; **, P<0.005; ***, P<0.001; ****, P<0.0001). The in vitro permeation results suggest that it is feasible to deliver phenmetrazine and 3-FA via transdermal route.

### Example 3—Chemical Permeation Enhancers

The effects of chemical enhancers on the permeation of phenmetrazine and of 3-flouroamphetamine was examined Fatty acid or fatty alcohol enhancers oleic acid, oleyl alcohol, and lauric acid where chosen as the tested permeation enhancers because the use of these enhancers are well documented and they are present in FDA-approved formulations of other drugs.

Chemical permeation enhancers belonging to fatty acid or fatty alcohol group, oleic acid, oleyl alcohol and lauric acid were incorporated to improve the permeation rate of phenmetrazine (40 mg/mL) and 3-flouroamphetamine (20 mg/mL).

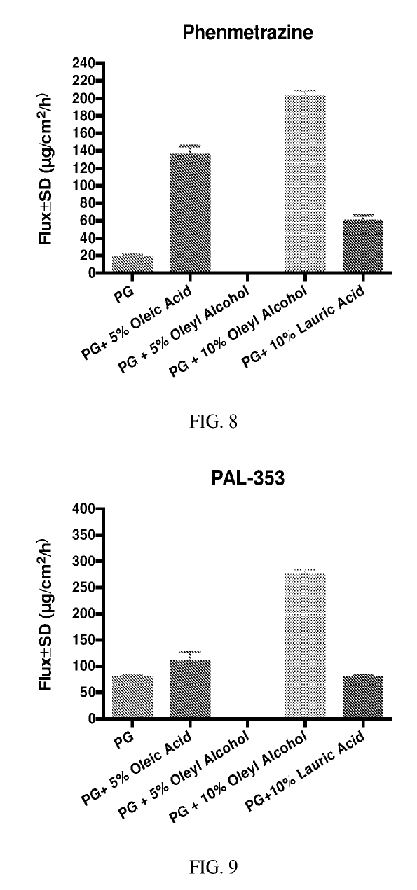

The permeation flux of both drugs with or without the enhancers is shown in FIGS. 8 and 9. For phenmetrazine, all the enhancers showed significantly (P<0.001 for lauric acid, P<0.0001 for others) higher flux compared to the 40-mg/mL group without the addition of enhancer, among which 5 and 10% oleyl alcohol equally increased the flux by ˜10 folds. Interestingly, although they have similar moieties other than their terminal groups, oleyl alcohol was 53.4% more efficient as a permeation enhancer for phenmetrazine than oleic acid at the same concentration (5% w/w). Compared to lauric acid, oleyl alcohol enhanced the flux by ˜240% irrespective of its concentration.

For 3-flouroamphetamine, oleyl alcohol (5% and 10% w/w) enhanced the flux significantly (P<0.0001). Oleic acid (5% w/w) enhanced the flux by ˜40%, but did not result in a significant difference as compared to PG alone. Interestingly, lauric acid did not impact the flux at all. The flux values from oleyl alcohol groups were 389% (5% w/w) and 370% (10% w/w) of the control group, while no significant difference was found between 5% and 10% w/w. (One-way ANOVA was used for statistics)

Chemical permeation enhancers were effective in modulating the transdermal delivery of both drugs, with oleyl alcohol being substantially more effective.

### Example 4—Development of a Transdermal Patch

Transdermal patch formulations allow for the non-invasive and continuous delivery of drug. Once absorbed, hepatic circulation is bypassed, thus avoiding another major site of potential degradation. However, there are physiochemical properties of many drugs that preclude transdermal formulation. Typically, only potent drugs can be administered through this route since there are economical and cosmetic reasons which restrict the patch size up to a certain limit. Though it is hard to make generalizations, the maximum patch size has been suggested to be about 50 cm². Furthermore, typically the drugs are moderately lipophilic (typically, log P of about 1 to 3) so as to be able to have significant passive permeation into the skin and then be able to diffuse out from the skin into the aqueous systemic circulation. Passive permeation is also generally considered to be limited to drugs with a MW less than 500 Da. 4-benzylpiperidine, PAL-353 (3-flouroamphetamine), and phenmetrazine have been found to possess physiochemical properties (as described earlier) amenable to transdermal formulation.

### Transdermal Patch Development:

As shown in FIG. 5, the formulation of a given drug with acrylate or another adhesive is mixed and then coated on a release liner by making a drawdown using a Gardner's casting knife. The prepared film is heated in an oven at required temperature for optimal duration to evaporate the organic solvent in which the acrylate adhesive is dissolved. The dried film is then laminated to a backing layer. Patches were die cut from this film for in vitro permeation testing and for characterization. The prepared patches were tested for in vitro permeation using human dermatomed skin in addition to physical properties such as thickness, weight, light microscopy, shear testing, tack testing and rheology. Based upon the physical properties of the patch and drug permeation, the addition of excipients and penetration enhancers were considered.

If a given compound showed limited passive or chemically enhanced skin permeation, physical enhancement technologies, including microneedles, laser, and iontophoresis, were utilized. If these technologies were found to be more successful, the daily patch was manufactured as more of a miniaturized wearable device that looked similar to a patch. The skin irritation potential of our formulations were tested using EPIDERM™, which is a 3 dimensional model used to assess skin irritation.

### Microneedles:

Skin microporation involved a minimally invasive technique in which transport pathways of microns dimension were created in the skin. These micron sized holes were tiny relative to the holes made by a hypodermic needle, but were huge relative to the hydrodynamic radius of even macromolecules. The holes were temporary since the stratum corneum was replaced through the natural process of desquamation.

Creation of micropores in human skin was demonstrated using maltose or other microneedles and characterized by a variety of methods. Creation of pores was demonstrated by staining with 1% (w/v) methylene blue dye solution and images were taken with a Proscope HR video microscope. Imaging were also prepared using calcein, a fluorescent dye with excitation/emission wavelengths of 495 nm/515 nm. Calcein images were processed using FLUOROPORE™ software, which measured fluorescent intensity in and around each pore to give a value called the pore permeability index (PPI). The PPI value is representative of calcein flux into the skin for each pore and therefore indicates the uniformity of the pores.

Transepidermal water loss (TEWL) measures any changes in barrier integrity and stratum corneum disruption by microneedles. TEWL readings were taken using a VapoMeter instrument to demonstrate the creation of the micropores. TEWL values of intact skin prior to treatment with microneedles were considered as baseline values.

### Iontophoresis:

Iontophoresis involved the application of a small amount of current to drive ionic compounds into the body. A physiologically acceptable current density in the range of 0.1-0.5 mA/cm² of the skin was used. By using an electrode of the same polarity as the charge on the compound, it was driven into the skin by electrostatic repulsion. The technique was found to be particularly well suited to deliver charged molecules, but even water soluble drugs were delivered by the electro-osmotic flow of water from anode to cathode. If possible, formulation pH was adjusted for delivery under anode so that electro-osmotic flow in the direction of anode to cathode facilitated transport. Silver/silver chloride electrodes were used to prevent electrolysis of water. Factors such as current density, or duration of application, were modulated to achieve desired flux rates.

The chloride content of the formulation was optimized to drive the electrochemistry of the silver/silver chloride electrodes without adding too many extraneous ions which may compete for the current. The rate of drug delivery was initiated, terminated or accurately controlled/modulated merely by switching the current on and off or adjusting the current application parameters. Performance was investigated across current density, delivery time, and dose titration.

### Laser Ablation:

Laser ablation involves the application of a high-energy laser to create microchannels through the stratum corneum. When the skin is exposed to the laser beam water molecules on the skin surface evaporates creating transport pathways in the skin. These micron sized holes are tiny relative to the holes made by a hypodermic needle, but were huge relative to the hydrodynamic radius of even macromolecules. The holes were temporary since the stratum corneum was replaced through the natural process of desquamation.

Erbium-YAG and CO₂ lasers have been used for transdermal delivery of macromolecules and/or vaccines, examples of systems used for laser ablation of skin to enhance skin permeability include but is not limited to the Precise Laser Epidermal System (P.L.E.A.S.E®, Pantec Biosolutions, Ruggell, Liechtenstein), the eCO₂™ (Lutronic, San Jose, Calif., USA), the UltraPulse® Fractional CO₂ Laser (Lumenis, Inc., Santa Clara, Calif., USA) and the Fraxel® CO₂ laser (Solta, Palo Alto, Calif., USA).

### Combination Enhancement Techniques:

A combination of microneedles and iontophoresis was also used to understand synergistic effects of combining these two technologies. Enhancement factors for iontophoretic delivery by varying current density (in the tolerable range) was compared for microporated vs. intact skin.

### Example 5—Microneedle Investigation

Microneedle enhanced delivery of PAL-353 across dermatomed human skin was also investigated.

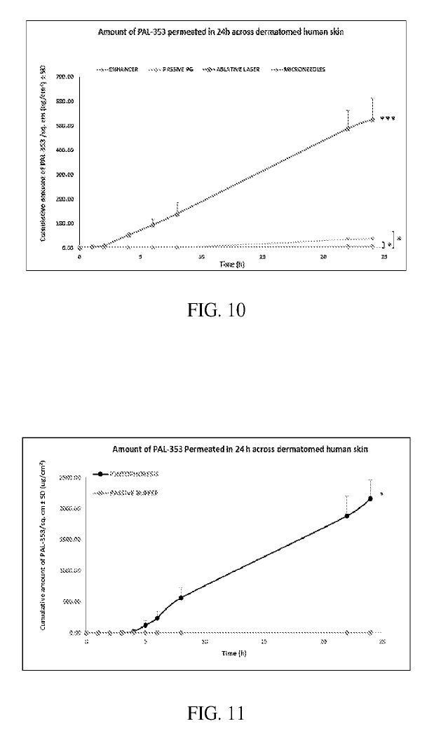

The permeation of PAL-353 (3-fluoroamphetamine hydrochloride) from its solution in propylene glycol (PG) as control, across dermatomed human skin after 24 h was observed to be 1.03±0.17 μg/cm². Pre-treatment of skin with maltose microneedles significantly increased the drug permeation to 7.35±4.87 μg/cm² as compared to the control (p<0.05), a 7-fold enhancement as shown in FIG. 10.

### Example 6—Iontophoresis Investigation

Iontophoretic delivery of PAL-353 across dermatomed human skin was also investigated. The donor chamber was filled with 500 μL of PAL-353 solution (10 mg/ml; 54% of saturation solubility) in 10 mM PBS, pH 7.4 containing 25 mM sodium chloride (n=4). The pH of the donor formulation as measured using glass electrode was around 4.0. Anodal iontophoresis was conducted where silver (anode) and silver chloride electrodes (cathode) were placed in the donor chamber and sampling port of the receptor chamber, respectively. It was ensured that there was no contact between the anode and skin in order to avoid skin damage due to high local voltage. The electrodes were then coupled in series to a source of constant current supply (Keithley 2400 Source Meter®, Keithley Instruments Inc., Cleveland, Ohio, USA). A current density of 0.5 mA/cm² was applied for 4 h. However, the total duration of the permeation study was 24 h and sampling was done at 0 h, 1 h, 2 h, 3 h, 4 h, 5 h, 6 h, 8 h, 22 h and 24 h.

This study was repeated keeping all the parameters same except that no current was applied (n=4). This group served as the passive control for comparison of the iontophoretic delivery of PAL-353 across human skin. Also, in a separate study, for these two groups, after 4 h, the formulations were removed and current was stopped in the iontophoresis group. Skin resistance was then measured using the procedure explained in section 2.2.3. Results have been presented as mean±SE.

Lag time was calculated as the x-intercept of the extrapolated linear portion of the permeation profiles (cumulative drug permeated/cm² plotted against the time).

A UV detection based reverse phase high performance liquid chromatography (RP-HPLC) was used for quantitative estimation of PAL-353. Waters Alliance 2695 separation module (Milford, Mass., USA) coupled with a 2996 photodiode array detector was used. Isocratic elution was performed on Kinetex 5μ EVO C18 100 A, 250*4.6 mm column (Phenomenex, CA, USA) at a flow rate of 1.0 ml/min and column temperature of 35° C. after injecting 30 μl of sample. The chromatographic conditions were: methanol (phase A) and 0.1% v/v TFA in DI water (phase B) in the ratio of 30:70. The run time was 10 min and the retention time of PAL-353 was around 4.7 min Drug standards were prepared in 10 mM PBS and detected at wavelength of 262 nm. The precision limit of detection and quantification were 0.02 μg/ml and 0.06 μg/ml, respectively and linearity was observed in the concentration range of 0.1-50 μg/ml (R²=0.9999). No interference due to the components leaching from the skin into the receptor was observed with the drug peak, while quantifying the amount of PAL-353 in the receptor using the above mentioned HPLC method.

Application of anodal iontophoresis (at 0.5 mA/cm² for 4 h) significantly increased the permeation of PAL-353 (2159.43±301.14 μg/cm²) in comparison to its passive permeation from PBS solution (3.94±0.53 μg/cm², p<0.05) as shown in FIG. 11. Lag time of around 3.5 h was observed. Iontophoretic treatment increased the cumulative drug permeation by 548 fold as compared to passive permeation over a 24 h period. Skin resistance after 4 h of current application was found to have decreased by 87.69±3.03%, that was significantly greater than in the control group (18.47±6.40%, p<0.05).

Findings of the present study were very interesting and significant enhancement in the transdermal delivery of PAL-353 was observed with the use of OA as chemical enhancer in the formulation as well as by physical enhancement techniques such as maltose microneedles, ablative laser, and anodal iontophoresis as compared to its passive permeation.

Microporation using ablative laser was investigated (n=4). Skin samples were placed on a flat platform (four layers of parafilm) and treated with P.L.E.A.S.E. (Precise Laser Epidermal System; Pantec Biosolutions AG, Liechtenstein). The treatment specifications included; fluence of 41.5 J/cm², 1.4 W, 10% density, array size of 8, and 3 pulses/pores. After laser treatment, skin pieces were mounted on the Franz cells for the in vitro permeation study. Application of skin microporation by ablative laser significantly increased the permeation of PAL-353 (523.24±86.79 μg/cm²) in comparison to its passive permeation from propylene glycol (PG) solution (1.03±0.17 μg/cm², p<0.05) as shown in FIG. 10. Anodal iontophoresis as well as skin microporation by ablative laser enhanced the skin permeation of PAL-353 manifolds (548 and 508 times, respectively), and were thus, found to be the most efficient permeation enhancing strategies for the transdermal delivery of the novel agent.

Findings also show that the passive permeation of 3-flouroamphetamine was approximately four-times higher in PBS as compared to PG, and that the total permeation of 3-flouroamphetamine in PBS enhanced by iontophoresis achieved was four-times higher as compared to enhancing permeation of 3-flouroamphetamine in PG by ablative laser treatment.

Iontophoresis, a physical enhancement technique, that drives charged or neutral drugs, into and through skin by application of a low constant current, works on the principle of electrorepulsion and electro osmosis. It was observed to be the most efficient technique for enhancing the transdermal delivery of PAL-353. Hydrochloride salt of 3-fluoroamphetamine was used in this study and being polar and water soluble, it was considered as a good candidate for iontophoresis. Moreover, 3-fluoroamphetamine is basic in nature with a pKa of 9.97 and at a pH of 4.0 (formulation pH), it would be positively charged and thus, anodal iontophoresis was used. Application of anodal iontophoresis resulted in the highest drug permeation amongst all the investigated physical and chemical enhancement techniques. Electroosmosis always occurs from anode to cathode and is also one of the mechanisms that contributes to the iontophoretic delivery of positively charged drug molecules. Permeation of PAL-353 was observed to increase linearly after termination of current that may be attributed to the change in the electrical properties of stratum corneum as evident by a significant drop in the skin resistance compared to the control group. Changes in the electrical properties of skin further indicated perturbation/disorganization of the stratum corneum barrier. This has been reported in earlier studies, where the effect of iontophoresis on the integrity of stratum corneum has been demonstrated with the help of FTIR, differential scanning calorimetry, transepidermal water loss, differential thermal analysis, freeze fracture electron microscopy, and XRAY diffraction studies. Anodal iontophoresis was thus, found to be the most effective strategy for enhancing the transdermal delivery of PAL-353 through dermatomed human skin.

It should be understood that although the above-described transdermal patches, pharmaceutical compositions and/or methods are described as “comprising” one or more components or steps, the above-described transdermal patches, pharmaceutical compositions and/or methods may “comprise,” “consists of,” or “consist essentially of” the above-described components, features or steps of the transdermal patches, pharmaceutical compositions and/or methods. Consequently, where the present invention, or a portion thereof, has been described with an open-ended term such as “comprising,” it should be readily understood that (unless otherwise stated) the description of the present invention, or the portion thereof, should also be interpreted to describe the present invention, or a portion thereof, using the terms “consisting essentially of” or “consisting of” or variations thereof as discussed below.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a transdermal patch, pharmaceutical composition and/or method that “comprises” a list of elements (e.g., components, features, or steps) is not necessarily limited to only those elements (or components or steps), but may include other elements (or components or steps) not expressly listed or inherent to the transdermal patch, pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a transdermal patch, pharmaceutical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

It is understood that derivatives of the active ingredients and salt forms of the active ingredients or chemical enhancers may be used in the present invention. According to the invention, suitable active ingredient or chemical enhancer can form a salt in ionic or anionic form with the respective substitute(s) with formation of positively or negatively charged forms. Typical salts include, hydrochloride, sodium, sulfate, acetate, phosphate, chloride, potassium, maleate, calcium, citrate, mesylate, nitrate, tartrate, gluconate, fumarate, epolamine and magnesium, for example. Additional examples of pharmaceutically acceptable salts include sulfates, pyrosulfates, bisulfates, sulfites, bisulfites, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, succinates, suberates, sebacates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenyl acetates, phenylpropionates, phenylbutyrates, citrates, lactates, γ-hydroxybutyrates, glycolates, tartrates, and mandelates. The invention also contemplates the use of other known pharmaceutically acceptable excipients for formulation.

As used herein, the term “pharmaceutical composition” contemplates compositions comprising one or more therapeutic agents or drugs as described above, and one or more pharmaceutically acceptable excipients, carriers, or vehicles.

As used herein, the term “pharmaceutically acceptable excipients, carriers, or vehicles” comprises any acceptable materials, and/or any one or more additives known in the art. As used herein, the term “excipients,” “carriers” or “vehicle” refer to materials suitable for drug administration through various conventional administration routes known in the art. Excipients, carriers, and vehicles useful herein include any such materials known in the art, which are nontoxic and do not interact with other components of the composition in a deleterious manner, and generally refers to an excipient, diluent, preservative, solubilizer, emulsifier, adjuvant, and/or vehicle with which an active agent or drug is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art.

As used herein, the term “therapeutically effective amount” refers to those amounts that, when administered to a particular subject in view of the nature and severity of that subject's condition, will have a desired therapeutic effect, e.g., an amount which will cure, prevent, inhibit, or at least partially arrest or partially prevent a target condition. In some embodiments, the term “therapeutically effective amount” or “effective amount” refers to an amount of a therapeutic agent or drug that when administered alone or in combination with an additional therapeutic agent or drug to a cell, tissue, or subject is effective to prevent or ameliorate conditions such as a narcolepsy, Attention Deficit Hyperactivity Disorder (ADHD), lethargy, appetite suppression, and substance-use disorders. A therapeutically effective dose further refers to that amount of the therapeutic agent or drug sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously.

As used herein, the terms “treating” or “treatment” or “alleviation” refers to therapeutic treatment wherein the object is to slow down (lessen) if not cure the targeted pathologic condition or disorder or prevent recurrence of the condition. A subject is successfully “treated” if, after receiving a therapeutic amount of a therapeutic agent or drug, the subject shows observable and/or measurable reduction in or absence of one or more signs and symptoms of the particular condition. Reduction of the signs or symptoms of a condition may also be felt by the patient. A patient is also considered treated if the patient experiences stable condition. In some embodiments, treatment with a therapeutic agent or drug is effective to result in the patients being symptom-free 3 months after treatment, preferably 6 months, more preferably one year, even more preferably 2 or more years post treatment. These parameters for assessing successful treatment and improvement in the condition are readily measurable by routine procedures familiar to a physician of appropriate skill in the art.

As used herein, “preventative” treatment is meant to indicate a postponement of development of a condition or a symptom of a condition, suppressing symptoms that may appear, or reducing the risk of developing or recurrence of a condition or symptom. “Curative” treatment includes reducing the severity of or suppressing the worsening of an existing symptom, or condition.

Further, it should be understood that the herein-described transdermal patches, pharmaceutical compositions and/or methods may comprise, consist essentially of, or consist of any of the herein-described components, features and steps, as shown in the figures with or without any feature(s) not shown in the figures. In other words, in some embodiments, the transdermal patches, pharmaceutical compositions and/or methods of the present invention do not have any additional features other than those shown in the figures, and such additional features, not shown in the figures, are specifically excluded from the transdermal patches, pharmaceutical compositions and/or methods. In other embodiments, the transdermal patches, pharmaceutical compositions and/or methods of the present invention do have one or more additional features that are not shown in the figures.

While the specification has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

1. A transdermal patch comprising: a substrate; and an effective amount of at least one drug on and/or within said substrate, said at least one drug comprising: (a) phenmetrazine; (b) 4-benzylpiperidine; (c) 3-flouroamphetamine, (d) a DA/NE releaser compound having formula (I): 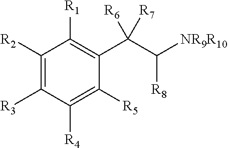
wherein each of R₁, R₂, R₃, R₄, R₅, R₆, R₇, R₈, R₉ and R₁₀ is independently hydrogen, a halogen, a substituted or unsubstituted alkyl group, or a substituted or unsubstituted alkoxy group, and when one or more substituents is present on an alkyl group, an alkoxy group, or both, each substituent is independently a halogen; or (e) any combination thereof.

2. The transdermal patch of claim 1, wherein said substrate comprises an adhesive layer.

3. The transdermal patch of claim 2, wherein said substrate further comprises a backing layer positioned on at least one major surface of said adhesive layer.

4. The transdermal patch of claim 3, wherein said backing layer comprises a film layer, a fibrous layer, or any combination thereof.

5. The transdermal patch of claim 3, wherein said substrate further comprises a release liner positioned on at least one major surface of said adhesive layer.

6. The transdermal patch of claim 3, wherein said adhesive layer comprises an acrylate (e.g., Duro-Tak 87-4098 and/or Duro-Tak 87-202A), a polyisobutylene (e.g., Duro-Tak 87-608A), a silicone, or any combination thereof.

7. The transdermal patch of claim 1, wherein said at least one drug is within said substrate.

8. The transdermal patch of claim 2, wherein said at least one drug is within said adhesive layer.

9. The transdermal patch of claim 1, wherein said at least one drug is phenmetrazine.

10. The transdermal patch of claim 1, wherein said at least one drug is 4-benzylpiperidine.

11. The transdermal patch of claim 1, wherein said at least one drug is a DA/NE releaser compound having formula (I).

12.-32. (canceled)

33. A transdermally applyable pharmaceutical composition, said composition comprising an therapeutically effective amount of at least one drug, said at least one drug comprising: (a) phenmetrazine; (b) 4-benzylpiperidine; (c) 3-flouroamphetamine; (d) a DA/NE releaser compound having formula (I) and at least one of R₁, R₂ and R₃ is a halogen and at least one of R₁, R₂ and R₃ is a substituted or unsubstituted alkyl group or a substituted or unsubstituted alkoxy group, (e) derivatives of drug (a) to (d), (f) salt forms of drug (a)-(e), or (f) any combination thereof.

34. The transdermal patch or transdermally applyable pharmaceutical composition of claim 33, wherein said effective amount of at least one drug comprises from greater than 0 to about 10 milligrams/kg/24 hour.

35.-40. (canceled)

41. A method of delivering one or more drugs to a patient, said method comprising:
transdermally administering an effective amount of at least one drug comprising: (a) phenmetrazine, (b) 4-benzylpiperidine, (c) 3-flouroamphetamine, (d) a DA/NE releaser compound having formula (I), or (e) any combination thereof, to the patient.

42.-43. (canceled)

44. The method of claim 41, wherein said transdermally administering step further comprises utilizing one or more enhancement techniques, the one or more enhancement techniques comprising use of microneedles, chemical enhancement, laser ablation, iontophoresis, or any combination thereof.

45. (canceled)

46. The method of claim 41, wherein said transdermally administering step further comprises utilizing one or more enhancement techniques, the one or more enhancement techniques comprising use of chemical enhancement, optionally selected from oleyl alcohol, isopropyl myristate, lauric acid, and combinations thereof.

47. The method of claim 41, wherein said transdermally administering step further comprises utilizing one or more enhancement techniques, the one or more enhancement techniques comprising use of laser ablation.

48. (canceled)

49. The method of claim 41, wherein said method is used to treat a patient with one or more disorders, the one or more disorders comprising narcolepsy, Attention Deficit Hyperactivity Disorder (ADHD), lethargy, appetite suppression, substance-use disorders (e.g., cocaine addiction), or any combination thereof.

50.-55. (canceled)

56. The method of claim 41, wherein the effective amount of at least one drug comprises from greater than 0 to about 10 milligrams/kg/24 hour.

57.-59. (canceled)

60. The transdermal patch of claim 41, wherein said transdermal patch is capable of delivering at least one drug over a period of time up to about 72 hours.

61.-68. (canceled)
